(19) United States
(12) Patent Application Publication
(10) Pub. No.: US 2013/0338482 A1
(43) Pub. Date: Dec. 19, 2013

(54) VIBRATION TYPE DRIVING DEVICE, MEDICAL APPARATUS, AND MEDICAL SYSTEM

(71) Applicant: CANON KABUSHIKI KAISHA, Tokyo (JP)

(72) Inventor: Yasumichi Arimitsu, Yokohama-shi (JP)

(21) Appl. No.: 13/916,350

(22) Filed: Jun. 12, 2013

(30) Foreign Application Priority Data
Jun. 15, 2012 (JP) 2012-135444

(51) Int. Cl. H02N 11/00; A61B 5/055; A61B 19/00

(52) U.S. Cl. CPC H02N 11/006; A61B 19/26; A61B 5/055
USPC 600/410; 310/300; 606/1

(57) ABSTRACT

A vibration type driving device comprising an electromechanical energy conversion element, an elastic member to which the electromechanical energy conversion element is attached, a driven member wherein a relative displacement is generated between the elastic member and the driven member, and a contact member between the elastic member and the driven member, wherein the elastic member is formed of non-metal material, the driven member is formed of non-metal material, and the contact member is formed of at least one of resin, non-oxide ceramics, and ceramics added with filler.

### BACKGROUND OF THE INVENTION

1. Field of the Invention

One aspect of the present disclosure relates to a vibration type driving device, for example, a vibration type driving device provided in the vicinity of or inside a gantry of the magnetic resonance imaging device, a medical apparatus including the vibration type driving device, or a medical system including the vibration type driving device.

2. Description of the Related Art

In recent years, a research and development of an operation support robot by image feedback with the use of a magnetic resonance imaging device has been actively performed in a medical support robot field. A diagnostic imaging is performed with a body surface of a patient covered with the gantry having a cylindrical shape in a traditional magnetic resonance imaging device. On the other hand, in recent years, an open magnetic resonance imaging device having a gantry with a large opening portion or a gantry with a large space at the center portion of the gantry has been developed, therefore, the possibility for intervention by an operation support robot or a doctor inside a magnetic resonance imaging device has been increasing. However, a magnetic resonance imaging device has a extremely strong static magnetic field of about 1.5 to 3.0 T and the magnetic field is controlled with extremely high degree of accuracy in order to determine information of three dimensional position accurately. Therefore, it is necessary to keep effect, such as image artifact, of the operation support robot or other medical equipments to image formation low. Therefore, the operation support robot or other medical equipments are required not to upset linearity and unity of magnetic field generated inside the magnetic resonance imaging device.

U.S. Pat. No. 6,274,965B1 discloses that, with respect to a vibration type driving device used in the vicinity of a magnetic resonance imaging device, structural elements except housing are formed of materials which do not affect image artifact of the magnetic resonance imaging device. U.S. Pat. No. 6,274,965B1 discloses that a structural example in which titanium, tantalum, or aluminum is used as the structural elements.

On the other hand, in “Kiyoyuki Chinzei, R. Kikinis, F. Jolesz, “MR Compatibility of Mechatronic Devices: Design Criteria,” in Proc. MICCAI'99 Lecture Notes in Computer Science, vol. 1679, 1999, pp. 1020-1031”, the experimental result that beryllium-copper having lower magnetic susceptibility than austenitic stainless steels such as SUS304 and SUS 316, metal such as aluminum and titanium which are generally said to be non-magnetic metal generates image artifact to the obtained image by a magnetic resonance imaging device.

Therefore, even if every structural elements of a vibration type driving device are formed of the material disclosed in U.S. Pat. No. 6,274,965B1, as a result, image artifact may be generated when the vibration type driving device is provided in the vicinity of a magnetic resonance imaging device.

### SUMMARY OF THE INVENTION

One aspect of the present disclosure is related to a vibration type driving device which generate no or reduced image artifact in a magnetic resonance imaging device when the vibration type driving device is provided in the vicinity of or inside the magnetic resonance imaging device.

For instance, one aspect of the present application is related to a vibration type driving device comprising an electromechanical energy conversion element, an elastic member to which the electromechanical energy conversion element is attached, a driven member wherein a relative displacement is generated between the elastic member and the driven member, and a contact member between the elastic member and the driven member, wherein the elastic member is formed of non-metal material, the driven member is formed of non-metal material, and the contact member is formed of at least one of resin, non-oxide ceramics, and ceramics added with filler.

Also, one aspect of the present disclosure is related to a medical apparatus comprising a medical equipment, a hold member that hold the medical equipment, and a vibration type driving device connected to the hold member wherein the vibration type driving device comprises an electromechanical energy conversion element, an elastic member to which the electromechanical energy conversion element is attached, a driven member wherein a relative displacement is generated between the elastic member and the driven member, and a contact member between the elastic member and the driven member, wherein the elastic member is formed of non-metal material, the driven member is formed of non-metal material, and the contact member is formed of at least one of resin, non-oxide ceramics, and ceramics added with filler.

Further, one aspect of the present disclosure is related to a medical system comprising a magnetic resonance imaging device and the above medical apparatus provided on the magnetic resonance imaging device.

Further features of the present invention will become apparent from the following description of exemplary embodiments (with reference to the attached drawings).

### BRIEF DESCRIPTION OF THE DRAWINGS

FIG. 1 is a brief diagrammatic perspective view of a vibration type driving device.

FIG. 2 is a brief diagrammatic perspective view of an exploded vibration type driving device.

FIG. 3A is a brief diagrammatic perspective view of a vibration type driving device, FIG. 3B is an enlarged view of B part of the vibration type driving device, and FIG. 3C is an enlarged view of C part of the vibration type driving device.

FIG. 4 is a brief diagrammatic perspective view of a vibration type driving device.

FIG. 5 is a brief diagrammatic perspective view of an exploded vibration type driving device.

FIG. 6A is a brief cross-sectional view and FIG. 6B is an enlarged view of D part of the vibration type driving device.

FIG. 7 is a brief diagrammatic perspective view of a vibration type driving device.

FIG. 8 is a brief diagrammatic perspective view of an exploded vibration type driving device.

FIG. 9A is a brief cross-sectional view and FIG. 9B is an enlarged view of D part of the vibration type driving device.

FIG. 10 is a brief diagrammatic perspective view of an open magnetic resonance imaging device.

FIG. 11 is a brief diagrammatic perspective view of a medical manipulator.

### DESCRIPTION OF THE EMBODIMENTS

An exemplary embodiment mode of the present disclosure will be described below.

One aspect of the present application is related to a vibration type driving device comprising an electromechanical energy conversion element, an elastic member to which the electromechanical energy conversion element is attached, a driven member wherein a relative displacement is generated between the elastic member and the driven member, and a contact member between the elastic member and the driven member, wherein the elastic member is formed of non-metal material, the driven member is formed of non-metal material, and the contact member is formed of at least one of resin, non-oxide ceramics, and ceramics added with filler.

Note that, in the subject specification, the expression “a member is formed of A” or “a member is formed from A” includes a case where the member is substantially formed of/from A in addition to a case where the member is formed of/from A. Therefore, the expression includes a case where the member comprises an element or material other than A as an impurity as long as the member has the same function as that of a member formed of/from A.

Also, a driven member is a member wherein a relative displacement is generated between the member and an elastic member due to a vibration of the elastic member. Note that the expression “a relative displacement is generated between an elastic member and a driven member” include a case where the elastic member and the driven member move, a case where the elastic member is fixed and the driven member moves, and a case where the driven member is fixed and the elastic member moves.

### First Embodiment Mode

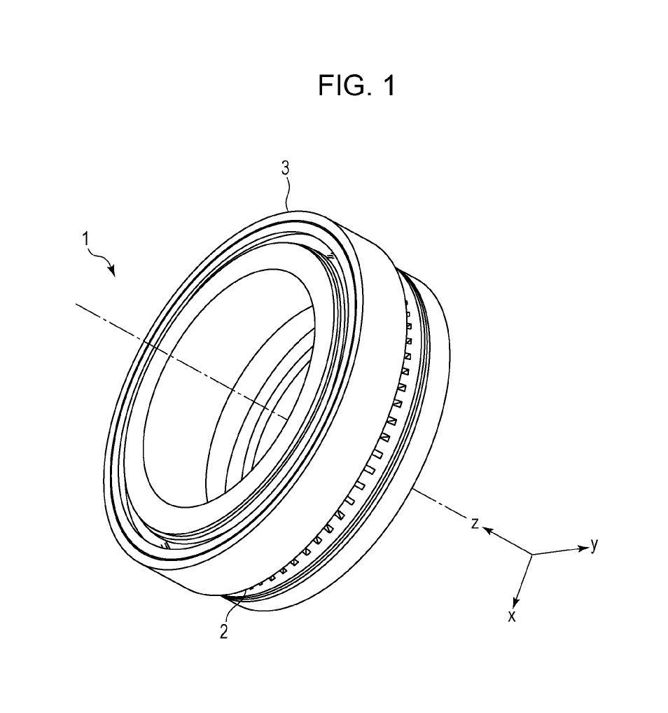

A first embodiment mode of the present disclosure is described with reference to FIGS. 1, 2, 3A, 3B, and 3C. Note that coordinate axes are common among the drawings.

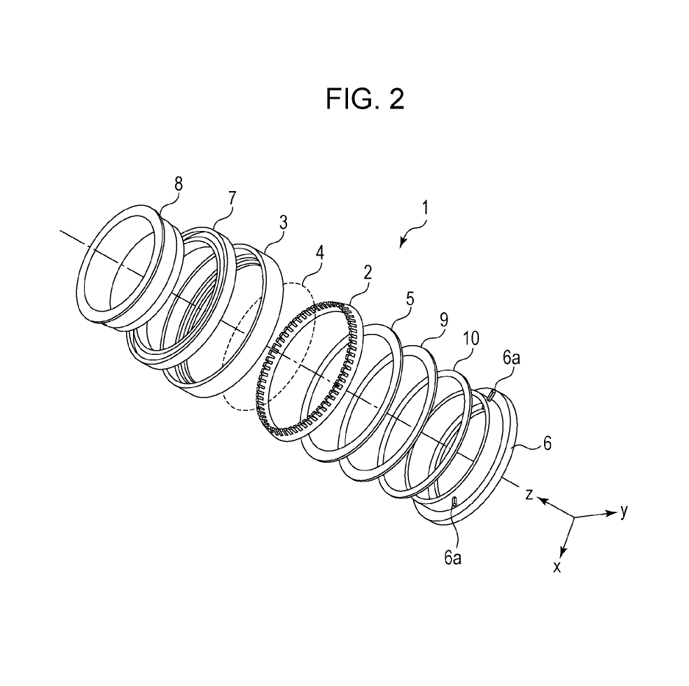

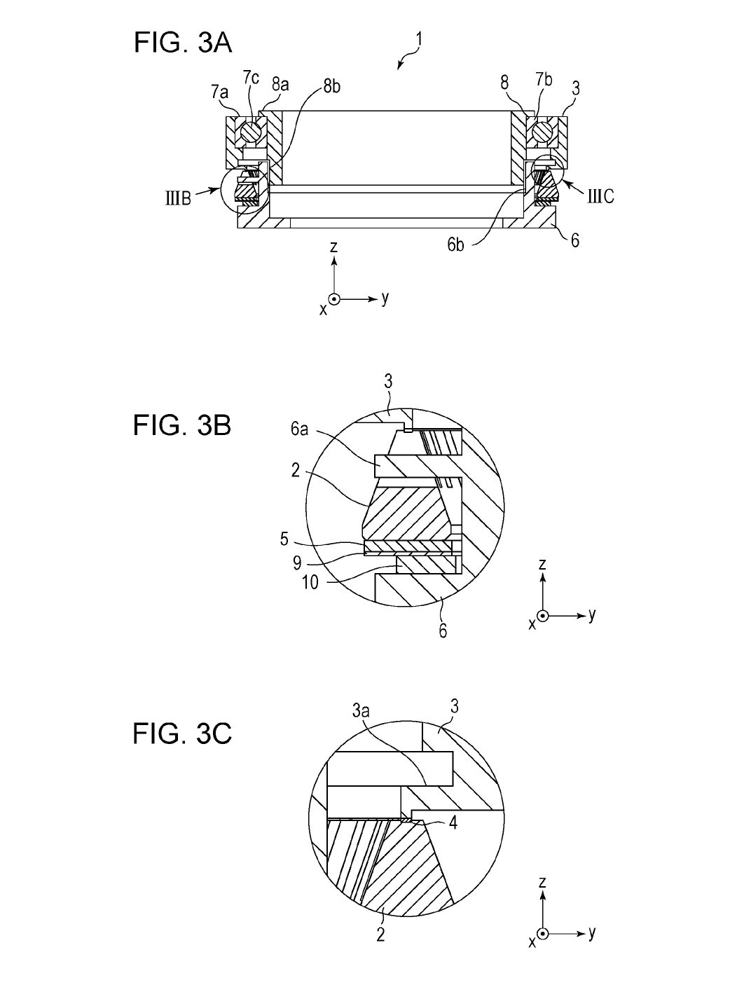

FIG. 1 is a brief diagrammatic perspective view of a vibration type driving device 1 in the first embodiment mode of the present disclosure. FIG. 2 is a brief exploded diagrammatic perspective view of the vibration type driving device shown in FIG. 1. Also, a cross-sectional view of the vibration type driving device shown in FIG. 1 along with y-z cross-section which include a center axis of the vibration type driving device, from the view of the positive side of an x-axis is shown in FIGS. 3A, 3B, and 3C. Enlarged views of IIIC part and IIIB part which are surrounded by circles in FIG. 3A are shown as FIG. 3B and FIG. 3C, respectively.

A structure and an operation principle of the vibration type driving device 1 are described. The reference number 2 denotes an elastic member. A mechanical energy providing element such as an electromechanical energy conversion element is provided on a rear surface of the elastic member 2. For example, a piezo element 5 is attached to the rear surface. When an electric signal is transmitted to the piezo element 5 via electric substrate 9, the piezo element 5 converts electrical energy to mechanical energy and generates a displacement in axial direction. A displacement in the axial direction and a displacement in a driving direction (circumferential direction in FIGS. 1, 2, 3A, 3B, and 3C) perpendicular to the axial direction can be obtained at a top surface of the elastic member 2 by polarization of the piezo element and excitation of a natural frequency corresponding to a bending vibration mode of the elastic member 2. Also, greater displacement can be efficiently obtained with lower energy by providing plurality of grooves on the elastic member 2 in a radial direction as shown in FIGS. 1, 2, 3A, 3B, and 3C. On the other hand, as shown in FIG. 3C, a groove is formed on a top end portion of the elastic member 2, and one end portion of a contact member 4 which is provided in the groove is attached to the elastic member 2. The other end portion of the contact member 4 is in contact with the driven member 3. The driven member 3 has an elastic portion 3a and the elastic portion 3a follows displacement of the elastic member 2 in the axial direction, therefore, the vibration type driving device has a structure with which the displacement in the driving direction (circumferential direction in FIGS. 1, 2, 3A, 3B, and 3C) can be efficiently taken from the displacement on the top end portion of the elastic member 2.

A support mechanism in the vibration type driving device 1 is described. The elastic member 2 is supported by a first support member 6. The first support member 6 is provided with hold mechanisms 6a in the radial direction with 120 degree intervals, and the hold mechanisms 6a are put in the grooves of the elastic member 2 as shown in FIG. 3B. Accordingly, the first support member 6 restricts and supports the elastic member 2 in the radial direction. A unit in which the electric substrate 9, the piezo element 5, and the elastic member 2 are integrated is held on an unwoven fabric 10 provided on the support member 6. While the hold mechanism 6a is shown as a part of the first support member 6 in the FIGS. 2 and 3B, the first support member may be formed with more than two bodies in consideration of the ease of embedding the hold mechanisms 6a. For example, the first support member 6 and the hold mechanisms 6a may be formed as separated members.

The way of supporting the driven member 3 is described. The reference number 7 denotes a second support member which supports the driven member 3. The second support member 7 is a radial ball bearing including an outer ring 7a, an inner ring 7b, and a plurality of balls 7c. The outer circumference portion of the outer ring 7a meshes with the inner circumference portion of the driven member 3 so that the driven member 3 is supported by the second support member 7. A top end portion of the inner ring 7b, and an elastic portion 8a provided in a pressure member 8 are in a contact condition, and an elastic deformation of the elastic portion 8b occurs by tightening a male screw 8b provided on an outer circumference portion and a female screw 6b provided on an inner circumference portion of the first support member 6. The pressure member 8 is a member to apply pressure to at least one of the elastic member 2 and the driven member 3 so that the at least one of the elastic member 2 and the driven member 3 is in contact with the contact member 4. The appropriate load in the axial direction can be applied to the contact member 4 and the at least one of the elastic member 2 and the driven member 3 by using the elastic deformation of the elastic portion 8b. As described above, a preferable frictional characteristics appropriate to the driving of the vibration type driving device 1 can be obtained by applying a pressure to the driven member 3 to the contact member 4 side with appropriate load.

The elastic member 2, the driven member 3, and the contact member 4 are formed of non-metal material in the vibration type driving device of this embodiment mode. For example, the elastic member 2 is formed of ceramics, the driven member 3 is formed of resin, and the contact member 4 is formed of resin or non-oxide ceramics. Note that in a case where the elastic member and the contact member are formed of ceramics, it may cause adverse affect on performance of the vibration type driving device due to instability of the frictional characteristics between the elastic member and the contact member caused by adsorption of moisture by the ceramics. Therefore, stable frictional characteristics can be obtained by forming the contact member with resin such as reinforced plastic (FRP), non-oxide ceramics such as silicon carbide, or ceramics added with non-oxide ceramics as filler, which have preferable tribological property.

Also, in addition to the above, the first support member 6 and the second support member 7, and the pressure member 8 are formed of non-metal material such as ceramics or resin.

The member except an electrode of the piezo element or a wiring of the electric substrate is formed of non-metal material in the vibration type driving device of this embodiment. Therefore, the vibration type driving device generates no disturbances or reduced disturbances in magnetic field when the vibration type driving device of this embodiment is used. Thus, the vibration type driving device can move or control a position of the object without disturbances or with reduced disturbances in magnetic field.

For example, in a case where the vibration type driving device of this embodiment mode is used in the vicinity of or inside a magnetic resonance imaging device, image formation with no influence or reduced influence can be performed in the magnetic resonance imaging device.

The specific example of materials for each structural members of the vibration type driving device in this embodiment mode is described. Firstly, material for the elastic member 2 is described. In this embodiment mode, ceramics or resin may be used as the material for the elastic member. As the resin, resin added with filler as reinforced plastic (ex. fiber reinforced plastic: FRP) may be used. In this embodiment mode, partially stabilized zirconia (PSZ) in which tetragonal zirconia is partially dispersed and deposited in cubic crystal and subjected to hot isostatic pressing (HIP) to be sintered. Stabilized zirconia (SZ) is used as heat resistance material. In the stabilized zirconia, magnesia (MgO), yttria (Y₂O₃), calcium oxide (CaO), or the like is added to zirconia (ZiO₂) and the mixture becomes solid solution, thereby the cubic crystal state is kept even at low temperature. On the other hand, since the partially stabilized zirconia (PSZ) includes cubic crystal as metastable phase, at least one of magnesia (MgO) and yttria (Y₂O₃) is added to zirconia (ZiO₂) with an lower amount than that is required for stabilizing zirconia in stabilized zirconia, then, a heat treatment is performed. These process can partially stabilized zirconia. Alumina (Al₂O₃) may be added the partially stabilized zirconia to be solid solution in order to increase strength. Using partially stabilized zirconia has an advantage of high toughness while it is fine ceramics, since energy possibly destroy zirconia at a crack tip in stress field is absorbed by martensitic transformation of zirconia from cubic crystal to monoclinic crystal. Also, alumina (Al₂O₃) may be used for the elastic member. For reference, performance comparison is conducted based on the simulation result with respect to a case where an elastic member is formed by using commercially available 99.5% alumina (Al₂O₃) and a case where an elastic member is formed by using partially stabilized zirconia (PSZ) in which yttria (Y₂O₃) is added to zirconia and sintered by hot isostatic pressing (HIP). The simulation is performed by assuming a case where a bending vibration mode having peak to peak amplitude of 2 micrometers is excited on an elastic member provided with grooves which has a width of 1.0 mm and a radius of flute of 0.2 mm wherein the elastic member has an outer diameter of 60 mm and a height of about 5 mm. Table 1 shows a simulation result of the calculation of maximum principal stress generated in the elastic member with respect to a case where the elastic member is formed by using commercially available 99.5% alumina (Al₂O₃) and a case where an elastic member is formed by using partially stabilized zirconia (PSZ). In table 1, the characteristic value for each material is brief reference value but not guaranteed value. From table 1, it can be understood that while the maximum principal stress is about 21% of the static deflective strength with respect to the elastic member formed using alumina, the maximum principal stress is about 2.6% of the static deflective strength with respect to the elastic member formed using partially stabilized zirconia (PSZ). The periodic bending vibration in the radial direction is repeatedly exited on the elastic member 2, therefore, it is preferable that the maximum principal stress is much lower than the static deflective strength generated in the elastic member. While it is difficult to determine a suitable ratio of a maximum principal stress to a deflective strength for elastic member since ceramics is a brittle material, Unwin's safety factor for brick or stone may be considered as a reference. The rough guideline of the Unwin's safety factor is said to be 30 when dynamic repeatedly stress is applied to the subject. Calculating from the simulation result in table 1, the Unwin's safety factors in a case of alumina and a case of partially stabilized zirconia (PSZ) corresponds to approximately 4.7 and approximately 37, respectively. Therefore, partially stabilized zirconia (PSZ) is considered to be a preferable material.

The partially stabilized zirconia has high specific gravity compared to other ceramics material such as resin or alumina (Al₂O₃) and the specific gravity of partially stabilized zirconia is approximately 79% of that of martensitic stainless steel, SUS420J2. Therefore, by using partially stabilized zirconia among fine ceramics as an elastic member, preferable vibration characteristics can be obtained among non-metal materials since higher vibration energy can be obtained and the viscosity loss is lower compared with the case of using resin.

TABLE 1

| | | Partially |
|---|---|---|
| | | stabilized |
| | 99.5% alumina | zirconia (PSZ) |
| Specific gravity | 3.9 | 6.09 |
| Young's modulus | 400 | 200 |
| [GPa] | | |
| Static deflective | 450 | 1700 |
| strength [MPa] | | |
| Maximum principal | 95 | 45 |
| stress [MPa] | | |

Material for the driven member 3 and a method for manufacturing the driven member 3 are described. As mentioned above, the driven member is required to have a stable elastic characteristics in a z-axis in FIGS. 1, 2, 3A, 3B, 3C, 4, 5, 6A, 6B, 7, 8, 9A, and 9B. Also, the natural frequency of the driven member corresponding to a vibration mode according to the bending vibration of the elastic member is required to be enough higher than the natural frequency corresponding to the bending vibration of the elastic member in order to realize preferable control characteristics of the vibration type driving device. In view of the above two points, the driven member may be formed by using resin added with filler and, for example, the driven member may be formed by using reinforced plastic (fiber reinforced plastic: FRP) in which glass fiber, carbon fiber, or the like is added to polyether ether ketone (PEEK). Glass fiber (GF) or carbon fiber (CF) added to the PEEK as filler functions as toughened wear-resistant material and also contributes to maintain the stable frictional force. In addition to the above, the material for the driven member may include fluorine resin such as polytetrafluoroethylene (PTFE) or heat resistant resin such as polyimide (PI) in order to improve tribological property. Also, ceramics or resin may include non-oxide ceramics such as silicon carbide (SiC), titanium carbide (TiC), or the like in order to improve frictional force. As the surface layer of the contact member, a coat formed of non-metal material such as diamond like carbon (DLC) or non-oxide ceramics such as silicon carbide (SiC) or titanium carbide (TiC), which is a toughened frictional layer may be formed. In a case where a driven member is formed by using commercially available reinforced plastic, it is common to form the driven member by cutting work and injection molding. However, since many of bars are formed by extrusion molding method, filler is aligned along the extrusion direction so that anisotropy in stiffness occurs. Further, the density of filler changes in the radial direction, therefore, an accidental error in characteristics can be easily occurs in the design. Also, in a case where an annular driven member is formed by injection molding method, inhomogeneous dispersion easily occur starting from the gate of metallic mold and the anisotropy in stiffness easily occurs. Therefore, in this embodiment mode, the driven member is formed by the following steps. At first, granular resin and fiber as filler are homogeneously mixed in advance, and compression molding is performed to the mixture during heating in the cylindrical metallic mold to form a blank with a cylindrical shaped (disk-shaped) which is larger than a driven member to be formed. Then, the blank formed by the compression molding is processed to be a predetermined size by machine processing such as turning process. The above steps realize uniform dispersion of the filler such as reinforced plastic, therefore, the elastic portion 3a can be designed with fine accuracy in elastic characteristics. As an example of the design, a case where the elastic portion 3a has a elastic function as the same as that in case of using aluminum alloy such as A5056 which is conventionally used, as a driven member is described. Polyether ether ketone added with 30% glass fiber has young's modulus which is 14% of that of aluminum alloy, A5056. When the PEEK added with 30% glass fiber is used as material for a driven member, the elastic characteristics similar to that of A5056 can be obtained by designing the size of the elastic portion 3a in z-axis in accordance with the driving frequency. In this manner, the advantage of improvement in the accuracy of elastic characteristic can be expected by inhomogeneous dispersion of filler. Further, expansion of creep time is expected by the addition of filler. In this embodiment, the example of the formation of the vibration type driving device, in which the granular resin and filler are mixed in advance and subjected to the compression molding to form the blank is described. However, even in a case where it is difficult to apply the above formation method, the anisotropy of a reinforced plastic (fiber reinforced plastic: FRP) including filler can be easily and briefly improved by the following method. The commercially available bar, pipe material, or other material is subjected to compression molding slowly during being heated in the metallic mold for the blank formation. With the above method, the advantage of disarraying the orientation of the alignment of filler is expected.

Then, the material for pressure member 8 is described. The pressure member 8 is provided with the elastic portion 8a. The elastic portion 8a is required to have high accuracy of elastic characteristics since the elasticity of the elastic portion 8a is used for controlling the precompression of the support member 7 and the contact pressure among the elastic member 2—contact member 4—driven member 3. For instance, the elastic portion 8a may be formed by using resin and the elastic characteristics with little anisotropy can be obtained when the pressure member 8 is formed by using general engineering plastic. Further, when a reinforced plastic (fiber reinforced plastic: FRP) including filler is used for the pressure member 8 similar to the case of the driven member 3, fine creep characteristics can be obtained.

Finally, materials for the piezo element 5, the first support member 6, the second support member 7, and the contact member 4 are described. As the piezo element 5, non-magnetic material such as ceramics can be used for the piezoelectric layer. For instance, the piezo element including lead zirconate and lead titanate (PbZrO₃—PbTiO₃) as main components can be used. Also, the first support member 6 can be formed by using resin or ceramics. Since the first support member 6 is not required to have high accuracy elastic characteristics or high heat resistance property, the first support member 6 may be formed by using non-metal material such as engineering plastic, machinable ceramics, and fine ceramics. With respect to the second support member 7, the outer ring 7a or the inner ring 7b may be formed by using resin or ceramics and non-metal material such as engineering plastic, machinable ceramics, and fine ceramics can be used. The ball 7c may be formed by using non-metal material such as ceramics. When the ball 7c is formed by using partially stabilized zirconia, the ball with high toughness, high heat resistance property, and high wear resistance can be formed. Also, the contact member 4 may be formed by using resin which is non-metal material. For the purpose of improving the tribological property, polytetrafluoroethylene (PTFE) which is one of fluorine resin may be preferably used as the material for the contact member 4. Further also, as resin, resin including filler such as ceramics may be used. For instance, resin including non-oxide ceramics such as silicon carbide (SiC) or titanium carbide (TiC) may be used. Alternatively, heat resistance resin such as polyimide (PI) may be included in resin. Also, ceramics may include non-oxide ceramics such as silicon carbide (SiC) or titanium carbide (TiC) in order to improve frictional force. As the surface layer of the contact member, a coat formed of non-metal material such as diamond like carbon (DLC) or non-oxide ceramics such as silicon carbide (SiC) or titanium carbide (TiC), which is a toughened frictional layer may be formed.

The materials for each member in this embodiment mode are described above. However, materials for a vibration type driving device of the present disclosure are not limited to the above description, and each member can be used as long as it is non-metal material. Note that the preferable tribological property can be obtained by forming a contact member with resin including fluorine resin such as polytetrafluoroethylene (PTFE) or heat resistant resin such as polyimide (PI) when an elastic member and a driven member are formed of ceramics. Also, the advantage of stable frictional characteristics is expected by adding non-oxide ceramics such as silicon carbide (SiC) or titanium carbide (TiC) to ceramics or resin to form a contact member.

Here, non-metal material means the material which is formed with a bonding other than the metal bonding, that is, the non-metal material means the material formed with ion bonding, covalent bonding, intermolecular force, or the like. Also, a phrase “a member is formed of A or formed from A” is not limited to the case where the member is formed of only A. The phrase includes the case where the member is substantially formed of A. Therefore, the embodiment mode include the case where a member includes unintended element or material with an amount with which image formation in a magnetic field, such as the image formation by a magnetic resonance imaging device, is not substantially affected.

In this embodiment mode, the piezo element which is an example of an electromagnetic energy conversion element is described as an example of the mechanical energy providing element. However, the present invention is not limited to the case. A mechanical energy providing element utilizing magnetostriction effect may be used. Also, the mechanical energy providing element is not limited to the conversion element which converts electric energy or magnetic energy to mechanical energy. The mechanical energy providing element may be a conversion element which converts energy of fluid, heat, or the like to mechanical energy can be used.

In this embodiment mode, the annular vibration type driving device which is a kind of a rotary type driving device is described. However, the concept of the present disclosure can be easily applied to a linear driving type, an in-plane driving type, and a spherical driving type driving devices in addition to a solid vibration type driving device which is a kind of a rotary type driving device.

### Second Embodiment Mode

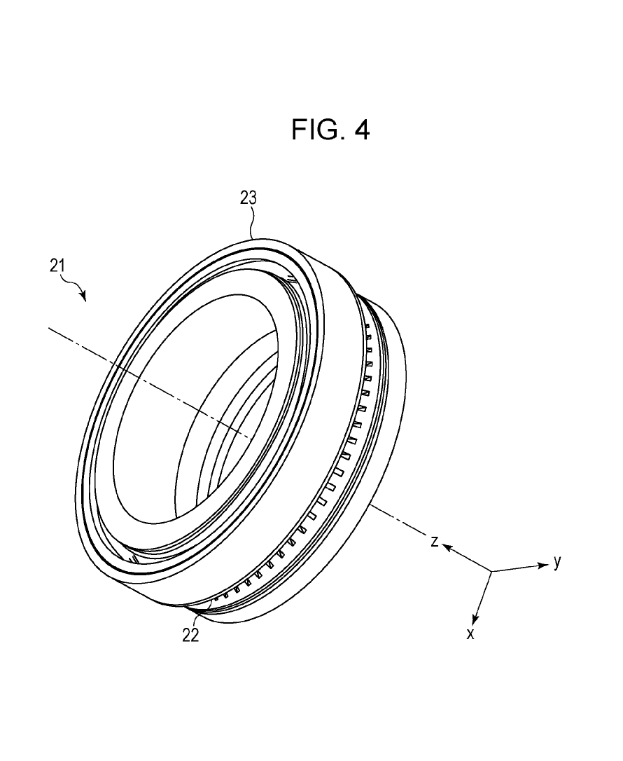

The second embodiment mode is described with reference to FIGS. 4, 5, 6A, and 6B. Note that axes shown in Figures are common. Also, the description is omitted to the common portion with the first embodiment mode, and the common part with the first embodiment mode is described with the same reference number.

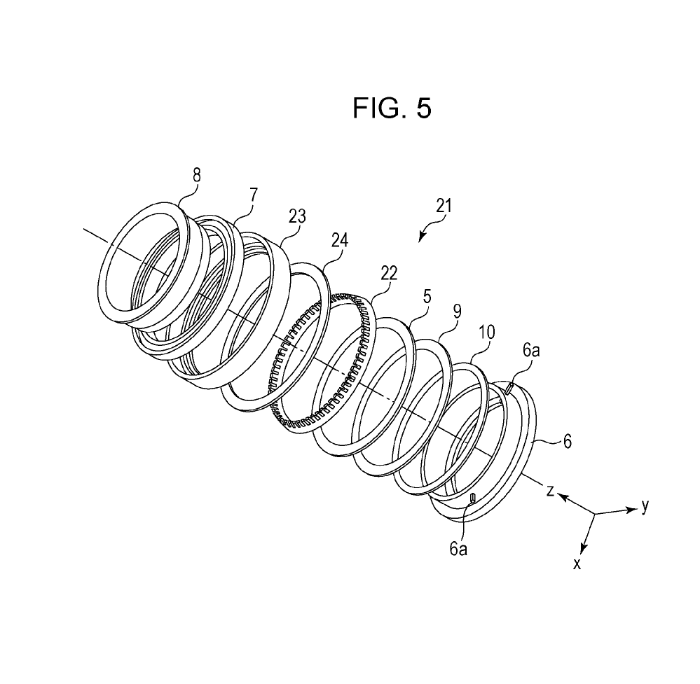

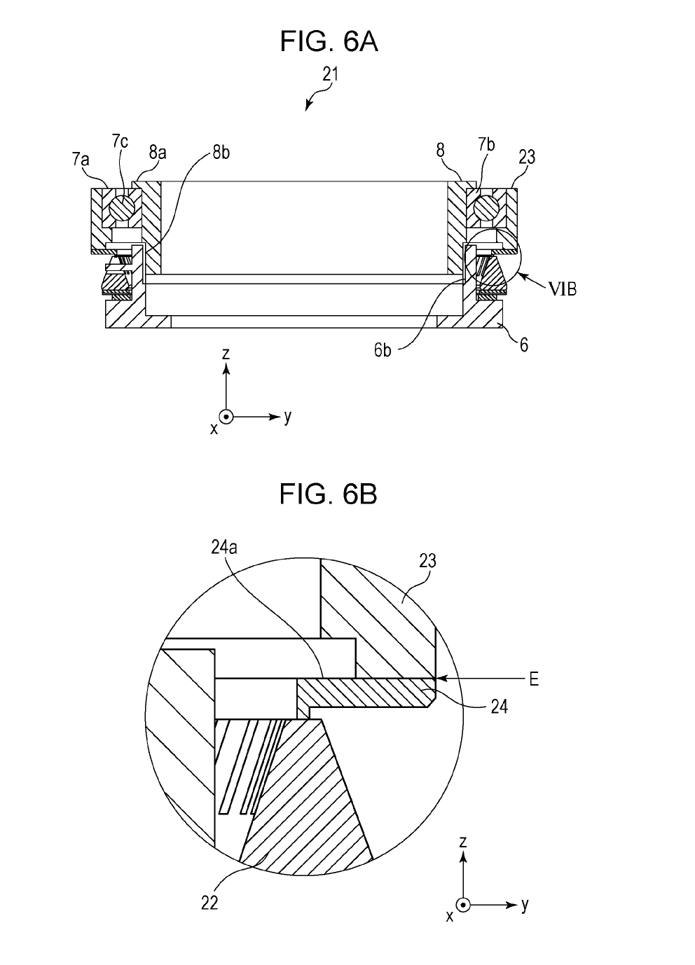

FIG. 4 is a brief diagrammatic perspective view of a vibration type driving device 21 in the second embodiment mode. FIG. 5 shows a brief diagrammatic perspective view of an exploded vibration type driving device 21. Also, FIG. 6A shows a brief cross-sectional view of the vibration type driving device shown in FIG. 4 along with y-z cross-section which includes a center axis of the vibration type driving device, from the view of the positive side of an x-axis. FIG. 6B is an enlarged view of IVB part surrounded by the circle in FIG. 6A.

A structure and an operation principle of the vibration type driving device 21 are described. The reference number 22 denotes an elastic member, and a mechanical energy providing element is provided on a rear surface of the elastic member 2. For example, a piezo element 5 which is an electromechanical energy conversion element is attached to the rear surface. Similar to the first embodiment mode, a displacement in an axial direction and a displacement in a driving axis (circumferential direction in FIGS. 4, 5, 6A, and 6B) perpendicular to the axial direction can be obtained at a top surface of the elastic member 22 by exciting the natural frequency corresponding to a bending mode of the elastic member 22. Also, a top end portion of a contact portion 24 is attached to a driven member 23 and a bottom portion of the contact portion 24 is in contact with a top end portion of the elastic member 22. The contact member 24 has an elastic portion 24a and the elastic portion 24a follows displacement of the elastic member 22 in the axial direction, therefore, the vibration type driving device has a structure with which the displacement in the driving direction (circumferential direction in FIGS. 4, 5, 6A, and 6B) can be efficiently taken out from the displacement on the top end portion of the elastic member 22.

A support mechanism in the vibration type driving device 21 is described. The elastic member 22 and the driven member 23 are supported by a first support member and a second support member 7, respectively. The supporting way is the same as that in the first embodiment mode.

The member except an electrode of the piezo element or a wiring of the electric substrate is formed of non-metal material in the vibration type driving device 21 of this embodiment. Therefore, the vibration type driving device generates no disturbances or reduced disturbances in magnetic field when the vibration type driving device of this embodiment is used. Thus, for example, in a case where the vibration type driving device of this embodiment mode is used in the vicinity of or inside a magnetic resonance imaging device, image formation with no influence or reduced influence can be performed in the magnetic resonance imaging device. The specific examples of non-metal materials in this embodiment mode are described below.

Firstly, material for the elastic member 22 is described. Ceramics or resin may be used as the material for the elastic member. In this embodiment mode, partially stabilized zirconia (PSZ) which is sintered by hot isostatic pressing (HIP) is used similar to the first embodiment mode.

Material for the driven member 23 is described. In this embodiment mode, the driven member is formed of ceramics. As mentioned in the first embodiment mode, the natural frequency of the driven member corresponding to a vibration mode according to the bending vibration of the elastic member is required to be enough higher than the natural frequency corresponding to the bending vibration of the elastic member in order to realize fine control characteristics of the vibration type driving device. Therefore, the driven member may be formed by using 99.5% alumina (Al2O3) so that the driven member has lower specific gravity and higher young's modulus than those of the elastic member.

The material for the contact member 24 is described. The contact member may be formed by using non-metal material such as resin. Regarding the contact member 24, the elastic portion 24a is required to have high accuracy elastic characteristics and the tribological property against the elastic member 22, resin including filler may be used as resin. For instance, the contact member 24 is formed by using a reinforced plastic in which filler such as glass fiber or carbon fiber is uniformly dispersed. As the formation method of the contact member 24, granular resin and the above filler are mixed and compression molding is performed to the mixture to form the contact member 24. As the resin, poly phenylene sulfide resin (PPS), polyether ether ketone (PEEK), or the like can be used. The glass fiber (GF) or carbon fiber (CF) added as filler functions as toughened wear-resistant material and also contributes to maintain the stable frictional force. Also, resin added with ceramics can be used as filler. For instance, resin including non-oxide ceramics such as silicon carbide (SiC) or titanium carbide (TiC) can be used in view of improvement of frictional force. In addition to the above, fluorine resin such as polytetrafluoroethylene (PTFE) or heat resistant resin such as polyimide (PI) may be included in order to improve tribological property. Also, ceramics including non-oxide ceramics such as silicon carbide (SiC), titanium carbide (TiC), or the like can be used in order to improve frictional force. As the surface layer of the contact member, a coat formed of non-metal material such as diamond like carbon (DLC) or non-oxide ceramics such as silicon carbide (SiC) or titanium carbide (TiC), which is a toughened frictional layer may be formed.

Finally, the materials for the piezo element 5, the electric substrate 9, the unwoven fabric 10, the first support member 6, the second support member 7, and the pressure member 8 can be used the same materials as those in the first embodiment mode.

In the vibration type driving device 21 of this embodiment, the driven member 23 has preferable controllability since the driven member 23 can be formed to have higher stiffness than that of the vibration type driving member in the first embodiment mode. Also, the contact member 24 may be formed of ceramics. Note that in a case the elastic member and a contact member are formed of ceramics, it may cause adverse affect on performance of the vibration type driving device due to instability of the frictional characteristics between the elastic member and the contact member, caused by adsorption of moisture by the ceramics. Therefore, by forming the elastic member 22 and the driven member 23 with ceramics and forming the contact member 24 with resin including filler, such as reinforced plastic (fiber reinforced plastic: FRP), high accuracy elastic characteristics is maintained and the frictional force can be maintained since the filler functions as toughened wear-resistant material. Also, favorable tribological property can be obtained when the driven device 24 includes fluorine resin such as polytetrafluoroethylene (PTFE) or heat resistance resin such as polyimide (PI). Further, the advantage of stable frictional characteristics can be expected by adding the toughened non-oxide ceramics such as silicon carbide (SiC) or titanium carbide (TiC) to ceramics or resin or forming coat formed of the materials.

In this embodiment mode, the driven member 23 is formed on one side of a surface which is parallel to an x-y plane shown by E in FIG. 6B, and the contact member 24 is formed on the other side of the surface. However, the driven member 23 and the contact member 24 may be adhered at different portion from the position E shown in FIG. 6B. In this embodiment mode, while the example in which the driven member 23 is formed of ceramics, a driven member may be formed of non-metal material such as reinforced plastic (fiber reinforced plastic: FRP). Also, the case where a driven member and a contact member are formed of the same material and a case where a driven member and a contact member are formed integrally (formed from the same member) are included in the scope of the present disclosure.

In this embodiment mode, the piezo element which is an example of an electromechanical energy conversion element is described as an example of the mechanical energy providing element. However, the present disclosure is not limited to the case. A mechanical energy providing element utilizing magnetostriction effect may be used. Also, the mechanical energy providing element is not limited to the conversion element which converts electric energy or magnetic energy to mechanical energy. The mechanical energy providing element may be a conversion element which converts energy of fluid, heat, or the like to mechanical energy.

Further, in this embodiment mode, the annular vibration type driving device which is a kind of a rotary type driving device is described. However, the concept of the present disclosure can be easily applied to a linear driving type, an in-plane driving type, and a spherical driving type driving devices in addition to a solid vibration type driving device which is a kind of a rotary type driving device.

### Third Embodiment Mode

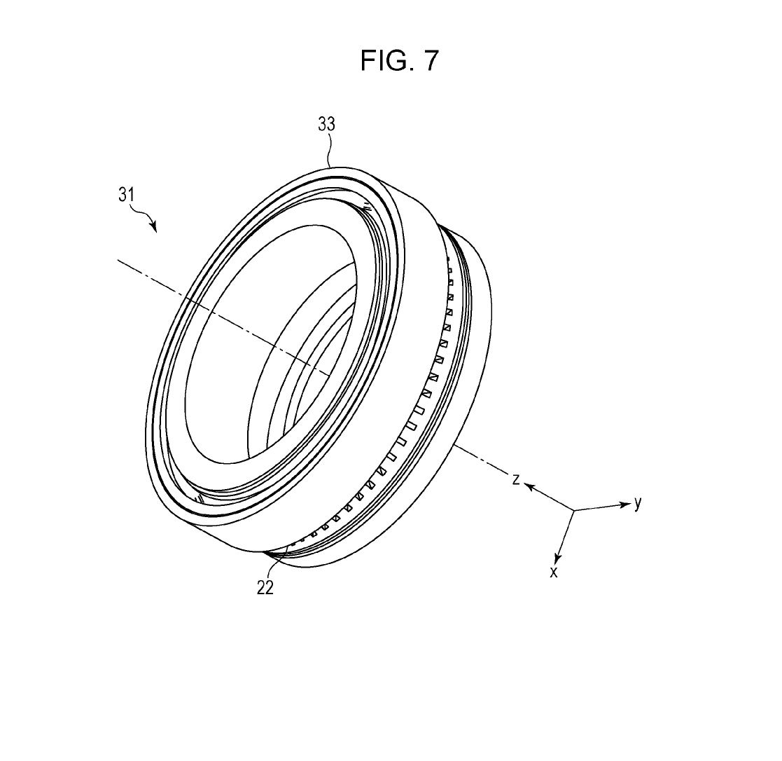

The third embodiment mode of the present application is described with reference to FIGS. 7, 8, 9A, and 9B. Note that axes shown in Figures are common. Also, the description is omitted to the common portion with the first embodiment mode or the second embodiment mode, and the common part with the first embodiment mode or the second embodiment mode is described with the same reference number.

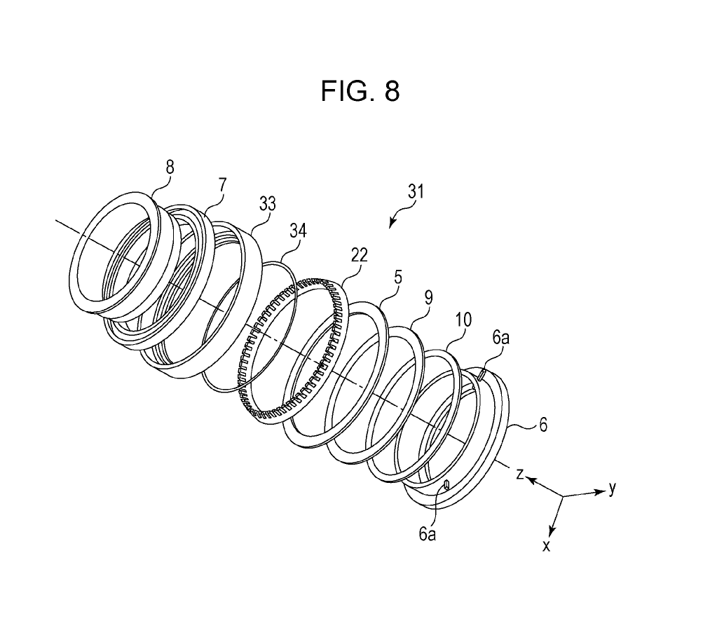

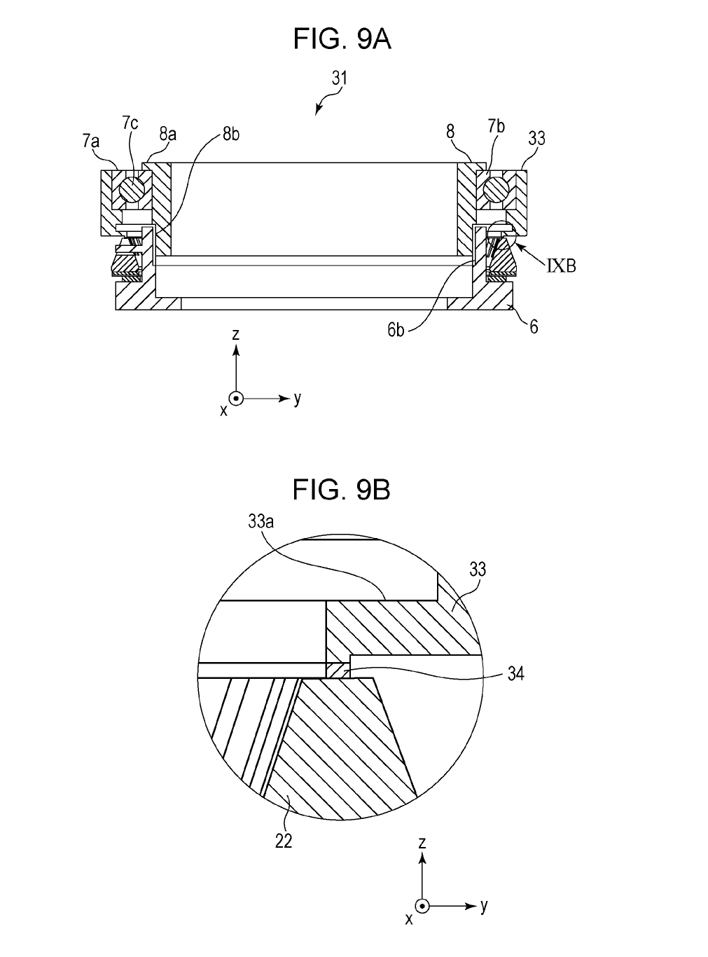

FIG. 7 is a brief diagrammatic perspective view of a vibration type driving device 31 of the third embodiment mode. FIG. 8 is a brief diagrammatic perspective view of a exploded vibration type driving device 31. FIG. 9A shows a brief cross-sectional view of the vibration type driving device shown in FIG. 7 along with y-z cross-section which include a center axis of the vibration type driving device, from the view of the positive side of an x-axis. FIG. 9B is an enlarged view of IXB part surrounded by the circle in FIG. 9A.

A structure and operation principle of the vibration type driving device 31 is described. The reference number 22 denotes an elastic member, and a mechanical energy providing element is provided on a rear surface of the elastic member 2. For example, a piezo element 5 which is an electromechanical energy conversion element is attached to the rear surface. Similar to the first embodiment mode, a displacement in an axial direction and a displacement in a driving axis (circumferential direction in FIGS. 7, 8, 9A, and 9B) perpendicular to the axial direction can be obtained at a top surface of the elastic member 22 by exciting the natural frequency corresponding to a bending mode of the elastic member 22. On the other hand, a top end portion of a thin film-like contact portion 34 is attached to a driven member 23 and a bottom portion of the thin film-like contact portion 34 is in contact with a top end portion of the elastic member 22. The driven member 33 has an elastic portion 33a and the elastic portion 33a follows displacement of the elastic member 22 in the axial direction, therefore, the vibration type driving device has a structure with which the displacement in the driving direction (circumferential direction in FIGS. 7, 8, 9A, and 9B) can be efficiently taken out from the displacement on the top end portion of the elastic member 22.

A support mechanism in the vibration type driving device 31 is described. The elastic member 22 and the driven member 23 are supported by a first support member and a second support member 7, respectively. The supporting way is the same as that in the first embodiment mode.

The member except an electrode of the piezo element or a wiring of the electric substrate is formed of non-metal material in the vibration type driving device 31 of this embodiment. Therefore, the vibration type driving device generates no disturbances or reduced disturbances in magnetic field when the vibration type driving device of this embodiment is used. Thus, for example, in a case where the vibration type driving device of this embodiment mode is used in the vicinity of or inside a magnetic resonance imaging device, image formation with no influence or reduced influence can be performed in the magnetic resonance imaging device. The specific examples of non-metal materials in this embodiment mode are described below.

Firstly, ceramics or resin may be used as the material for the elastic member 22. In this embodiment mode, partially stabilized zirconia (PSZ) which is sintered by hot isostatic pressing (HIP) is used for the elastic member 22 similar to the first embodiment mode.

The driven member 33 is formed of partially stabilized zirconia (PSZ) which is one of ceramics and has high toughness.

The contact member 34 may be formed of resin, non-oxide ceramics, or ceramics to which non-oxide ceramics is added as filler. For instance, the contact member 34 may be formed by using fluorine resin such as polytetrafluoroethylene (PTFE) in order to improve tribological property. Also, heat resistant resin such as polyimide (PI) may be included in the contact member 34. When non-oxide ceramics such as silicon carbide (SiC), titanium carbide (TiC), or the like is included in ceramics or resin, frictional force can be improved. Also, as the surface layer of the contact member, a coat formed of non-metal material such as diamond like carbon (DLC) or non-oxide ceramics such as silicon carbide (SiC) or titanium carbide (TiC), which is a toughened frictional layer may be formed.

Also, the materials for the piezo element 5, the electric substrate 9, the unwoven fabric 10, the first support member 6, the second support member 7, and the pressure member 8 can be used the same materials as those in the first embodiment mode.

In the vibration type driving device 31 of the present disclosure, the driven member 33 can be formed to have high toughness, which maintain s the preferable elastic characteristics of the driven member 33. The elastic member 22 and the driven member 33 are formed of partially stabilized zirconia (PSZ). Therefore, when the elastic member 22 and the driven member 33 are in direct contact with each other, it may cause adverse affect on performance of the vibration type driving device due to instability of the frictional characteristics by adsorption of moisture by the partially stabilized zirconia. Hence, preferable tribological property can be obtained by providing the contact member 34 between the elastic member 22 and the driven member 33 as described in this embodiment mode.

In this embodiment mode, the example in which the driven member 33 is formed of ceramics is describe d. However, the driven member may be formed of reinforced plastic (fiber reinforced plastic: FRP) which is a kind of non-metal material. The contact member is preferably formed of polytetrafluoroehylene (PTFE). Also, the contact member may include heat resistant resin such as polyimide (PI). Also, the contact portion may be formed of ceramics or resin, to which non-oxide ceramics such as silicon carbide (SiC) or titanium carbide (TiC) is added in order to improve frictional force. Also, as the surface layer of the contact member, a coat formed of non-metal material such as diamond like carbon (DLC) or non-oxide ceramics such as silicon carbide (SiC) or titanium carbide (TiC), which is a toughened frictional layer may be formed. Further also, the case where a driven member and a contact member are formed of the same material and a case where a driven member and a contact member are formed integrally (formed from the same member) are included in the scope of the present disclosure.

In this embodiment mode, the piezo element which is an example of an electromagnetic energy conversion element is described as an example of the mechanical energy providing element. However, the present disclosure is not limited to the case. A mechanical energy providing element utilizing magnetostriction effect may be used. Also, the mechanical energy providing element is not limited to the conversion element which converts electric energy or magnetic energy to mechanical energy. The mechanical energy providing element may be a conversion element which converts energy of fluid, heat, or the like to mechanical energy.

Further, in this embodiment mode, the annular vibration type driving device which is a kind of a rotary type driving device is described. However, the concept of the present disclosure can be easily applied to a linear driving type, an in-plane driving type, and a spherical driving type driving devices in addition to a solid vibration type driving device which is a kind of a rotary type driving device.

### Fourth Embodiment Mode

An application example of a vibration type driving device of the present disclosure is described in this embodiment mode. A vibration type driving device of the present disclosure can be used for a medical apparatus used inside a magnetic resonance imaging device. Also, a vibration type driving device of the present disclosure can provide a medical system including a medical apparatus which is capable of being used inside a magnetic resonance imaging device.

A medical system is provided with a medical apparatus including a vibration type driving device of the present disclosure. In this embodiment mode, the medical apparatus including a medical equipment, a hold member which holds the medical equipment and is configured to be movable, and a vibration type driving device which is connected to the hold member and moves the hold member. With this structure, the medical procedure can be performed with the medical apparatus while the image information regarding patient is being obtained by a magnetic resonance imaging device.

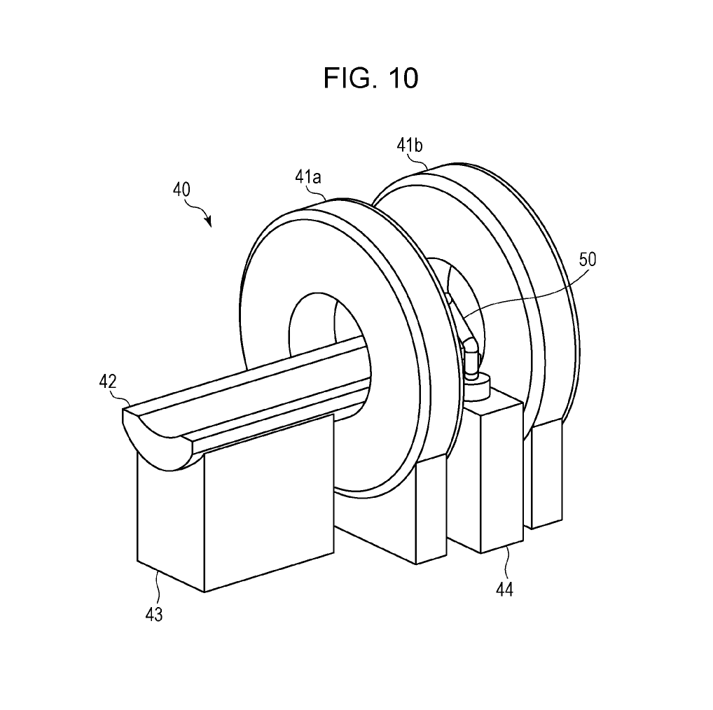

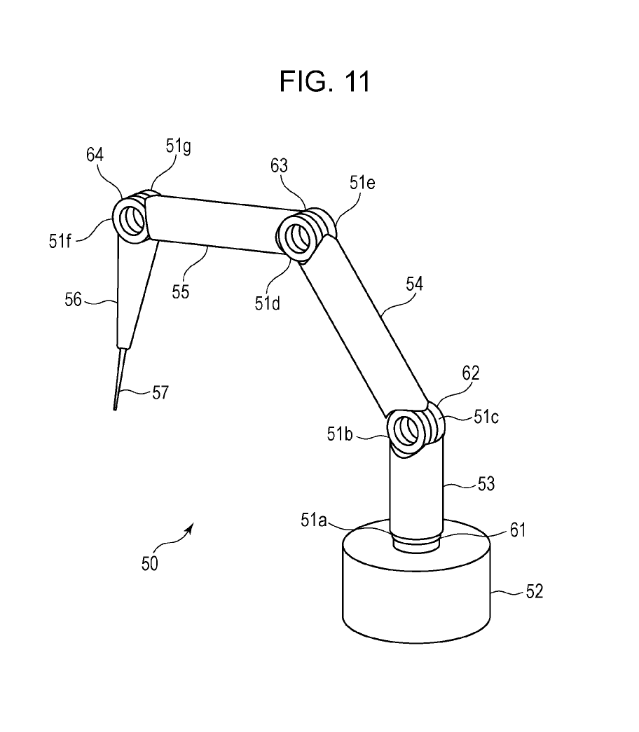

An example where a medical manipulator, an arm, and an end effector are used as a medical apparatus used inside a magnetic resonance imaging device, as a fix portion, and as a medical equipment, respectively is described with reference to FIGS. 10 and 11. Note that the medical equipment is not limited to the above, and include knife, forceps, needle, probe, and equipments for diagnosis, for example.

FIG. 10 is a brief diagrammatic perspective view which schematically shows a magnetic resonance imaging device 40 provided with a medical manipulator 50 of the present application. Here, a double donuts type open magnetic resonance imaging device is described as a magnetic resonance imaging device. In FIG. 10, the reference number 42 denotes a medical table for laying patient, the reference number 43 denotes a hold portion of the medical table 42, and the reference numbers 41a and 41b each denote a magnet which is a part of the magnetic resonance imaging device, and the magnet has a cylindrical shape. A manipulator set-up table 44 on which the medical manipulator 50 is set up is provided between the cylindrical magnet 41a and the cylindrical magnet 41b. Since the medical manipulator 50 is provided between the cylindrical magnet 41a and the cylindrical magnet 41b, the medical procedure can be performed with the medical manipulator 50 while the image information regarding patient is being obtained by a magnetic resonance imaging device.

The detailed structure of the medical manipulator 50 is described with reference to FIG. 11. The medical manipulator 50 has a 4-axis vertical multi-joint type arm structure in which a first joint 61, a second joint 62, a third joint 63, and a fourth joint 64 are provided between a manipulator base 52 and a first arm 53, the first arm 53 and a second arm 54, the second arm 54 and a third arm 55, the third arm 55 and a fourth arm 56, respectively. Each of the first joint 61, the second joint 62, the third joint 63, and the fourth joint 64 has a single-degree-of-freedom. Each of vibration type driving devices 51a, 51b, 51c, 51d, 51e, 51f, and 51g includes a first support member supporting an elastic member and the like and a second support member supporting a driven member and the like, as described in the first embodiment mode, the second embodiment mode, or the third embodiment mode. The first joint 61, the second joint 62, the third joint 63, and the fourth joint 64 are provided with the vibration type driving device 51a, the vibration type driving devices 51b and 51c, the vibration type driving devices 51d and 51e, and the vibration type driving devices 51f and 51g, respectively, hence, the driving torque can be provide on each of the first to fourth joints 61, 62, 63, and 64. Also, an end effector 57 is mounted on an end portion of the fourth arm 56 which is a hold member, the end effector 57 performs an arbitral medical procedure such as puncture, cauterization, grip, or the like.

The installation of the vibration type driving devices 51a to 51g is described. A first support member and a second support member of the vibration type driving device 51a are connected to the manipulator base 52 and the first arm 53, respectively, hence the vibration type driving device 51a has a structure capable of providing rotary torque around the first joint 61. First support members and second support members of the vibration type driving devices 51b and 51c are connected to the first arm 53 and the second arm 54, respectively, hence the vibration type driving devices 51b and 51c each have a structure capable of providing rotary torque around the second joint 62. First support members and the second support members of the vibration type driving devices 51d and 51e are connected to the second arm 54 and the third arm 55, respectively, hence the vibration type driving devices 51d and 51e each have a structure capable of providing rotary torque around the third joint 63. First support members and second support members of the vibration type driving devices 51f and 51g are connected to the third arm 55 and the fourth arm 56, respectively, hence the vibration type driving devices 51f and 51g each have a structure capable of providing rotary torque around the fourth joint 64.

The material for the medical manipulator 50 is described. Any one of the vibration type driving devices in the first embodiment mode, the second embodiment mode, and the third embodiment mode may be used for any one of the vibration type driving devices 51a, 51b, 51c, 51d, 51e, 51f, and 51g. Therefore, the members except an electrode and a wiring may be formed of non-metal material. Also, the manipulator base 52, the first arm 53, the second arm 54, the third arm 55, and the fourth arm 56 are formed of non-magnetic material such as non-metal material or non-magnetic metal material like berylium cupper.

Therefore, a medical manipulator can be provided in the vicinity of a cylindrical magnet of a magnetic resonance imaging device. Further, a medical manipulator with reduced effect of a magnetic resonance imaging device to magnetic field can be provided. Hence, artifact in the obtained image of a magnetic resonance imaging device can be suppressed. Also, the response of a medical manipulator can be improved due to the reduction of a power transmission structure such as a gear or a belt by directly providing a vibration type driving device of the present disclosure to a joint of the medical manipulator. Further also, the driving torque can be compensated by providing a plurality of vibration type driving devices of the present disclosure on a joint of a medical manipulator.

This embodiment mode shows an example in which a medical manipulator is provided inside a double donuts type open magnetic resonance imaging device as a magnetic resonance imaging device is described. However, a magnetic resonance imaging device is not limited to the above example. Also, while the example of the manipulator having the 4-axis vertical multi-joint type arm structure is described as a medical manipulator, a medical manipulator having a horizontal multi-joint type arm structure or a parallel link manipulator can be used, and the degrees of freedom or the location or the number of vibration type driving devices are not limited. Also, the example of the medical manipulator in which the vibration type driving device which is one kind of the rotary driving type is directly provided on the joint is described, however, the vibration type driving device is not limited to this example. A linear driving type, an in-plane driving type, and a spherical driving type driving devices may be used. Further, a power transmission structure may be provided as a providing unit of power torque to a joint.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2012-135444, filed Jun. 15, 2012, which is hereby incorporated by reference herein in its entirety.

What is claimed is:

1. A vibration type driving device comprising:
an electromechanical energy conversion element;
an elastic member to which the electromechanical energy conversion element is attached;
a driven member wherein a relative displacement is generated between the elastic member and the driven member due to excitation of vibration to the elastic member; and
a contact member between the elastic member and the driven member,
wherein the elastic member is formed of a non-metal material,
wherein the driven member is formed of a non-metal material, and
wherein the contact member is formed of at least one of resin, non-oxide ceramics, and ceramics added with filler.

2. The vibration type driving device according to claim 1, wherein the driven member is formed of a resin.

3. The vibration type driving device according to claim 1, wherein the driven member is formed of reinforced plastics.

4. The vibration type driving device according to claim 1, wherein the driven member is formed of ceramics.

5. The vibration type driving device according to claim 1, wherein the elastic member is formed of a resin.

6. The vibration type driving device according to claim 1, wherein the elastic member is formed of reinforced plastics.

7. The vibration type driving device according to claim 1, wherein the elastic member is formed of ceramics.

8. The vibration type driving device according to claim 1, wherein the contact member is a part of the driven member and formed of a same material as that of the driven member.

9. The vibration type driving device according to claim 1, wherein the contact member is a part of the elastic member and formed of a same material as that of the elastic member.

10. The vibration type driving device according to claim 1, further comprising:
a first support member that supports the elastic member, the first support member being formed of a non-material material,
a second support member that supports the driven member, the second support member being formed of a non-metal material,
a pressure member that applies pressure to at least one of the elastic member and the driven member so that the at least one of the elastic member and the driven member is in contact with the contact member.

11. A medial apparatus comprising:
a medical equipment;
a hold member that holds the medical equipment; and
a vibration type driving device connected to the hold member, the vibration type driving device comprising:
an electromechanical energy conversion element:
an elastic member to which the electromechanical energy conversion element is attached;
a driven member wherein a relative displacement is generated between the elastic member and the driven member due to excitation of vibration to the elastic member; and
a contact member between the elastic member and the driven member,
wherein the elastic member is formed of a non-metal material,
wherein the driven member is formed of a non-metal material, and
wherein the contact member is formed of a non-metal material.

12. The medial apparatus according to claim 11, wherein the driven member is formed of a resin.

13. The medial apparatus according to claim 11, wherein the driven member is formed of reinforced plastics.

14. The medial apparatus according to claim 11, wherein the driven member is formed of ceramics.

15. The medial apparatus according to claim 11, wherein the elastic member is formed of a resin.

16. The medial apparatus according to claim 11, wherein the elastic member is formed of reinforced plastics.

17. The medial apparatus according to claim 11, wherein the elastic member is formed of ceramics.

18. The medial apparatus according to claim 11, wherein the contact member is formed of a resin.

19. The medial apparatus according to claim 11, wherein the contact member is formed of at least one of non-oxide ceramics and ceramics added with filler.

20. The medial apparatus according to claim 11, wherein the contact member is a part of the driven member and formed of a same material as that of the driven member.

21. The medial apparatus according to claim 11, wherein the contact member is a part of the elastic member and formed of a same material as that of the elastic member.

22. The medial apparatus according to claim 11,
wherein the vibration type driving device further comprises:
a first support member that supports the elastic member, the first support member being formed of a non-metal material,
a second support member that supports the driven member, the second support member being formed of a non-metal material,
a pressure member that applies pressure to at least one of the elastic member and the driven member so that the at least one of the elastic member and the driven member is in contact with the contact member.

23. A medical system comprising:
a magnetic resonance imaging device; and
the medical apparatus according to the claim 11 provided on the magnetic resonance imaging device.

24. The medial apparatus according to claim 11, wherein the medial equipment is at least one from the group consisting of an end effector, knife, forceps, needle, probe, and an equipment for diagnosis.
